(19) United States
(12) Patent Application Publication
(10) Pub. No.: US 2009/0192423 A1
(43) Pub. Date: Jul. 30, 2009

(54) Therapeutic massaging device

(76) Inventor: Tracy Lea Halmos, Morgan Hills, CA (US)

Correspondence Address: TY UNG, 16830 VENTURA BLVD., SUITE 360, ENCINO, CA, 91436, US

(21) Appl. No.: 12/070,642

(22) Filed: Feb. 19, 2008

Related U.S. Application Data
(60) Provisional application No. 60/919,569, filed on Mar. 23, 2007.

(51) Int. Cl. A61H 7/00; A61B 17/00

(52) U.S. Cl. 601/134; 606/204

(57) ABSTRACT

A massaging device for relieving the stiffened muscles of a user. The device comprises a strap comprising a plurality of pockets disposed along its length. The device further comprises a plurality of balls that are to be received within the plurality of pockets. When the strap is secured around a desired body part, the balls keep pressed against the body part so as to produce a massaging effect thereon.

### CROSS-REFERENCE TO RELATED APPLICATION

This application claims priority to provisional application No. 60/919,569, filed on Mar. 23, 2007.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates in general to massaging devices and more particularly to a therapeutic massaging device made up of a strap embedded with a plurality of balls along its length. The device produces a massaging effect on a desired body part by exerting localized pressure thereon. It may also function as a lumbar support for an office chair, car, or couch. If strapped around the hips, it acts as a sacroiliac joint stabilization belt. Furthermore, it can function to correct rib dysfunctions and aid the lumbar spine or for core strengthening exercise.

Several massaging devices are known to exist in the art. For example, U.S. Pat. No. 6,007,501 to Cabados discloses a therapeutic massaging apparatus comprising a pair of resilient balls adjustably positioned in a flexible container, and a strap assembly connected to the opposite ends of the container so as to maintain the container in a pre-selected position upon the user. The container includes a pocket within which, other therapeutic principals are inserted for utilizing the benefits of traditional acupressure therapy.

U.S. Pat. No. 5,736,213 to Meier discloses a lotion applicator and massaging device comprising an elongated body attached with elastic handles on opposite ends thereof. Balls may be inserted in the body for producing a massaging effect.

U.S. Pub. No. 20040006292 to Joseph discloses a pressure point sleeve and balls device comprising a cylindrical main body within which, a plurality of balls are accommodated. The balls can be added from both ends of the main body. To make use of the device, a desired body part is made to abut and press against the cylindrical main body inserted with the balls. This produces localized pressures on the body part, which in turn produce a massaging effect thereon.

It is an object of the present invention to provide a hands-free therapeutic massaging device that can produce a massaging effect on the body by exerting localized pressures.

A further object is to provide a therapeutic massaging device that employs a plurality of balls embedded in a strap. This eliminates the necessity of constantly adjusting the balls with respect to the position of the body as the strap is secured around the body.

A further object is to provide a therapeutic massaging device comprising a strap made of a strong, triple reinforced, non-elastic material so as to make sure that the balls are firmly pressed against the body as the strap is secured there around. These and other objects of the present invention will become better understood with reference to the appended Summary, Description, and Claims.

### SUMMARY

The present invention is a therapeutic massaging device comprising a plurality of balls embedded into a strap along its length. Both sides of the strap are lined with Velcro™ for securing the strap around a desired body part. As the strap is secured around the body, the balls keep pressed against the body so as to produce a desired massaging effect thereon.

### BRIEF DESCRIPTION OF THE FIGURES

FIG. 1 is a top plan view of the therapeutic massaging device in accordance with the present invention.

FIG. 2 is a bottom plan view of the therapeutic massaging device in accordance with the present invention.

FIG. 3 is a side view of the therapeutic massaging device in accordance with the present invention.

FIG. 4 is a perspective view of the therapeutic massaging device in accordance with the present invention.

FIG. 5 is a side view of the therapeutic massaging device inserted with the balls in accordance with the present invention.

FIG. 6 is a perspective view of the therapeutic massaging device inserted with the balls in accordance with the present invention.

FIG. 7 is an illustration of the side view of the therapeutic massaging device worn by a user.

### FIGURES—REFERENCE NUMERALS

10 . . . Therapeutic Massaging Device
12 . . . Strap
14 . . . Hand Loop
16 . . . Velcro™
18 . . . Elastic Strip
20 . . . Ball

### DETAILED DESCRIPTION

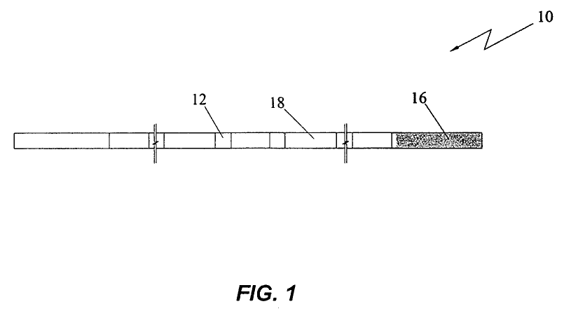

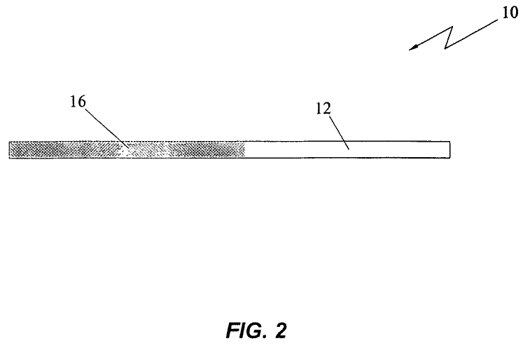

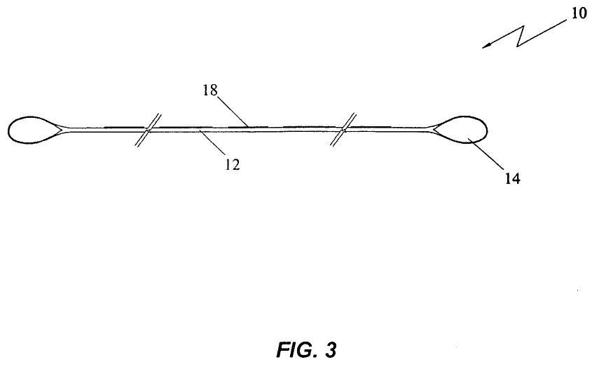

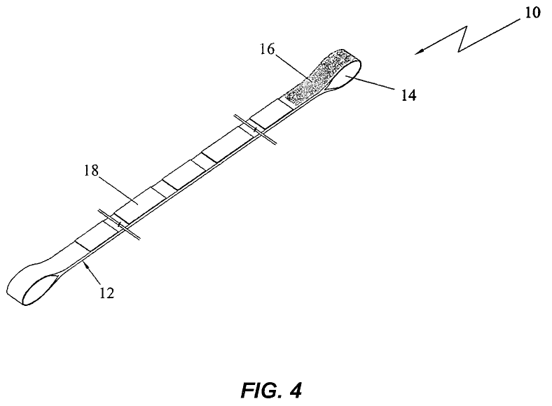

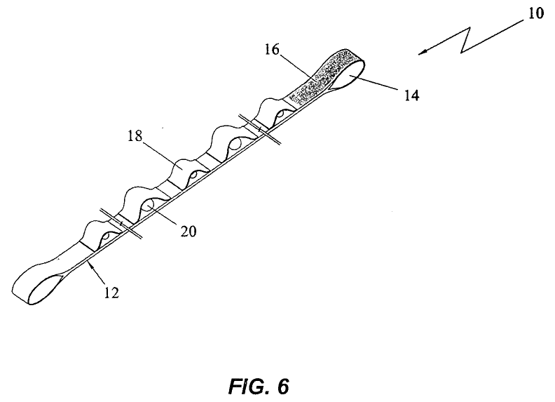

Referring to the drawings, a preferred embodiment of a therapeutic massaging device is illustrated and generally indicated as 10 in FIGS. 1 through 7. The device 10 is for relaxing tightened/stiffened muscles of a user by exerting localized pressures thereon. The device 10 can be operated without user of external power sources such as batteries, and so on. The operation of the device 10 is easy and can be carried out by a user alone. The device 10 can be employed at anytime, such as while driving, working, and so on.

Referring to FIGS. 1 through 4, the device 10 comprises a strap 12 and a plurality of balls 20 that are embedded into a strap 12 along its length. The strap 12 is made of a strong, triple-reinforced, non-elastic material. Both sides of the strap 12 are lined with Velcro™ 16 for securing the strap 12 around a desired body part such as the thigh, waist, shoulder, and so on. The ends of the strap 12 terminate in hand loops 14 about which the device 10 is to be handled. The strap 12 further comprises a plurality of pockets disposed along its length. The pockets are for receiving the balls 20. Each pocket is formed by attaching the opposite ends of the elastic strip 18 to the strap 12 so as to form a loop. The strips 18 abut the strap 12 due to the tension. The strap 12 and the elastic strips 18 are of the same width. The strips 18 are made of an elastic material called spandex.

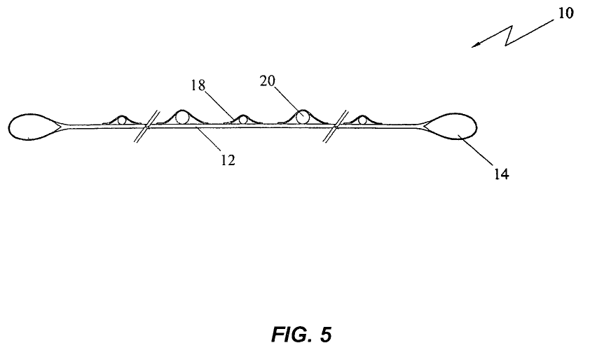

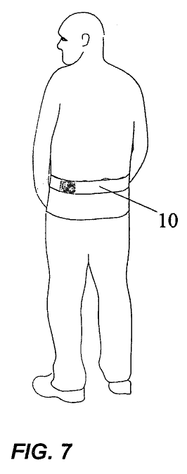

Referring to FIGS. 5 through 7, elastic strips 18 ensure that the balls 20 are firmly held in place once they are received within the pockets. The strips 18 are also wide enough to prevent any spillage of the balls 20. The strong, triple reinforced, non-elastic material of the strap 12 forces the balls 20 to be projected entirely in the direction of the rectangular strips 18 as the balls 20 are received within the pockets. When the strap 12 is secured around a desired body part, as seen in FIG. 7, the balls 20 push into the body and thus produce a desired massaging effect thereon.

Still referring to FIGS. 5 through 7, the balls 20 employed are replaceable and are of different sizes and resiliencies. The diameters of the balls 20 range between 0.8 inches and 6 inches. For example, tennis balls can be received within the pockets. Accordingly, the pockets are made of various sizes to accommodate the various-size balls 20. Two large pockets are disposed on the strap 12 centrally with a small pocket disposed between them. The large pockets receive big balls 20, while the small pocket receives a small ball 20. This arrangement is designed for gluteal placement of the strap 12. Also, the strap 12 further comprises two medium-sized pockets, each located near the hand loop 12 as seen in the figure. The medium-sized pockets are for receiving medium-sized balls 20. It is noteworthy that the strap can hold up to seven balls.

An additional embodiment of the present invention (not shown) comprises a strap comprising a plurality of pockets disposed along its length. The pockets can be closed on all the sides once the balls are received there within.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. A therapeutic massaging device comprising:
(a) a strap;
(b) a plurality of pockets disposed on the strap along its length; and
(c) a plurality of balls received within the pockets.

2. The device of claim 1, wherein the strap is made of a strong, triple-reinforced, non-elastic material.

3. The device of claim 1, wherein the balls are replaceable.

4. The device of claim 1, wherein the pockets are disposed on one side of the strap.

5. The device of claim 1, wherein each pocket is formed by attaching the opposite ends of a strip to the strap so as to form a loop.

6. The device of claim 5, wherein the strips are made of an elastic material.

7. The device of claim 6, wherein the strips are made of an elastic material called spandex.

8. The device of claim 6, wherein the strips abut the strap due to tension.

9. The device of claim 5, wherein the strap and strips are equal in width.

10. The device of claim 1, wherein the pockets can be closed once the balls are received thereinto.

11. The device of claim 1, wherein the pockets are of different dimensions for holding balls of different sizes.

12. The device of claim 11, wherein one small and two large pockets are located centrally on the strap such that the small pocket is disposed between the large pockets.

13. The device of claim 1, wherein the balls are of different sizes and resiliencies.

14. The device of claim 13, wherein the diameter of the balls vary between 0.8 inch and 6 inches.

15. The device of claim 1, wherein both ends of the strap terminate in hand loops, with which the device is to be handled.

16. The device of claim 1, wherein both sides of the strap is lined with Velcro™ for securing the strap around a desired body part.

17. The device of claim 1, wherein the strap can hold up to seven balls.

18. The device of claim 1, wherein the balls comprise tennis balls.

19. A method of using a therapeutic massaging device comprising:
(a) a strap comprising a plurality of pockets disposed along its length; and
(b) a plurality of balls disposed along its length; the balls to be received within the pockets; the method comprising fastening the strap around a desired body part such that the balls are pressed against the body part thereby producing a massaging effect thereon.

20. A method for relieving the stiffened muscles of a user, the method comprising the steps of:
(a) providing a strap comprising a plurality of pockets disposed along its length, the strap made of a strong, triple-reinforced material;
(b) providing a plurality of balls that are to be received within the pockets; and
(c) fastening the strap around a desired body part such as the thigh, waist, shoulder, and so on, such that the balls are pressed into the body part.
